(19) United States
(12) Patent Application Publication
(10) Pub. No.: US 2010/0317898 A1
(43) Pub. Date: Dec. 16, 2010

(54) NOVEL CATALYST FOR ALDOL CONDENSATION REACTIONS

(75) Inventors: Barbara Noziere, Tyreso (SE); Armando Cordova, Stockholm (SE)

Correspondence Address: YOUNG & THOMPSON, 209 Madison Street, Suite 500, Alexandria, VA, 22314, US

(73) Assignee: Barbara NOZIERE, Tyreso (SE)

(21) Appl. No.: 12/680,747

(22) PCT Filed: Sep. 29, 2008
(86) PCT No.: PCT/SE08/51091

§ 371 Date: Aug. 17, 2010

(30) Foreign Application Priority Data
Oct. 1, 2007 (SE) 0702203-1

(51) Int. Cl. C07C 45/45

(52) U.S. Cl. 568/388; 568/458

(57) ABSTRACT

Present invention presents catalytic systems for aldol condensation reactions. The efficiency of the catalytic systems of the present invention is comparable to those of the classical strong acid or strong base catalysts.

### FIELD OF THE INVENTION

The present invention relates to aldol condensation reactions, and more specifically to inorganic ammonium salts or solutions thereof, which can be used for catalyzing such reactions.

### BACKGROUND OF THE INVENTION

Aldol condensation reactions, or aldolization reactions, are known in the art. See U.S. Pat. No. 6,090, 986 to Godwin et al. See also Mestres et al, Green Chem., 2004, 6, 583-603. Ketones and/or aldehydes are starting materials for the aldol condensation, which is a convenient way to form C-C bonds, and is important for e.g. bulk production, or in the fine chemical and pharmaceutical industry.

Aldol condensation reactions are typically performed in the presence of strong acids or bases, and/or metal catalysts. A drawback with these processes is their costs, for the metal catalysts, and the negative impact on the environment for the strong acid/strong base catalysts used in bulk production. A further drawback is provided by the harsh conditions imposed by the strong acids or bases, which means that not all types of starting materials can be used. More environment-friendly productions are provided by using heterogeneous catalysis or phase transfer catalysis, but these are complex to implement and (at least in large scale) expensive processes.

Two types of aldol condensation reactions frequently encountered are the self-aldol condensation (Aldol I) and cross-aldol condensation (Aldol II) reactions.

In an Aldol I reaction, two molecules of the same aldehyde or ketone starting material react to form a reaction product. Alternatively, in an Aldol II reaction, two different aldehydes or ketones starting materials react to form a reaction product. In practice, the condensation of two molecules of aldehyde or ketone to form an aldol is usually followed immediately by dehydration to form an unsaturated aldehyde or ketone with twice the original number of carbon atoms. Both Aldol I and Aldol II reactions are well known in the art, as are the conditions required to effect their condensation.

### SUMMARY OF THE INVENTION

The present invention relates to a process for forming aldol condensation products, which comprises reacting at least one aldehyde or ketone starting material in the presence of an inorganic ammonium salt or a solution thereof. The solution may be an aqueous solution or an organic solution.

The use of an inorganic ammonium salt or an aqueous solution thereof is cheap (cheaper than organic, metal-based, and heterogenous catalysts), especially for ammonium sulfate, (NH₄)₂SO₄, which is by-product of other industrial processes. Inorganic ammonium salts can be used in the same liquid-phase reactors as currently used for strong base or strong acid catalysts and have a catalytic efficiency equivalent to (in some cases better than) the strong base or strong acid catalysts. Inorganic ammonium salt solutions, and in particular ammonium sulfate solutions, (NH₄)₂SO₄, have the advantage of providing a more environmentally friendly process (milder pH range) than strong acids or strong bases, which would significantly reduce the costs in waste treatment. In addition, inorganic ammonium salt solutions allow for gentler reaction conditions (milder pH) than previously known processes. The method allows the use of substrates with sensitive functional groups. For example, the aldol reaction is compatible with substrates containing ester and carbonate functionalities.

As starting materials can ketones and/or aldehydes be used. Examples of commonly used aldehydes include but are not limited to: formaldehyde, acetaldehyde, propionaldehyde, n-butyraldehyde, isobutyraldehyde or other branched aldehydes (substituted methyl-, ethyl-), aldehydes containing small cycles (C5-C6), containing aromatic groups such as benzaldehydes, glyoxylates, pyruvates, and mixtures of any two of them, although other aldehydes are also suitable.

Examples of ketones commonly used include but are not limited to: acetone and other aliphatic ketones (butanone, pentanone), substituted ketones (methyl-, ethyl-), cyclic ketones such as cyclopentanone and cyclohexanone, hydroxyacetone, dihydroxyacetone, and aromatic ketones (phenyl ketones). Large organic molecules might have a limited solubility in aqueous solutions of ammonium salts, in which case organic solutions containing these salts might be preferred. An example of substrate widely used in the bulk industry is acetone, for which aldol condensation gives mesityl oxide, the first step of the production of methyl isobutyl ketone (MIBK), which is very much in demand in the industry.

In another embodiment, the invention provides a device for use in the preparation of compounds formed by aldol condensation. Such a device may be a container for reactions run in batch mode, or may be a container for reactions run in continuous flow mode.

### DETAILED DESCRIPTION OF THE INVENTION

The phrase “aldol condensation” is used herein to refer to the process whereby an aldehyde and/or ketone starting materials are reacted and, upon immediate dehydration, form aldol condensation products. Aldol I and Aldol II are the terms used to label two types of aldol condensation.

The term “catalyst” is used herein to include all forms of catalysts, including classic initiators, co-initiators, co-catalysts, activating techniques, etc. Preferably, the term “catalyst” includes inorganic ammonium salts or aqueous or organic solutions thereof. The solution is preferably aqueous. In a preferred embodiment, the aqueous inorganic ammonium solution is prepared from NH₄F, (NH₄)₂SO₄, NH₄NO_{3,} NH₄Cl, NH₄Br, NH₄I, (NH₄)₂CO_{3,} (NH₄)HSO₄, (NH₄)HCO_{3,} and, in general all inorganic salts containing NH₄⁺cations. A preferred inorganic ammonium solution, which is low cost and environmentally friendly, is prepared from ammonium sulfate, (NH₄)₂SO₄.

The present invention describes a new method to carry out aldol condensation reactions. By utilizing inorganic ammonium salts or solutions thereof as a catalyst, it is possible to perform aldol condensation as a cheap, simple, process and use more sensitive substrates. In addition, using aqueous solutions of inorganic ammonium salts is more environmentally friendly than current processes using strong basic or strong acidic conditions, organic solvents, or metal-based catalysts, which would lower both the production costs (no special reactor or safety measures required) and waste treatment costs (neutralization of acids or bases). The efficiency of inorganic ammonium salts is comparable to those of the classical strong acid or strong base catalysts (see comparison in Table 1), with some variations depending on the initial carbonyl compounds. For acetone, inorganic ammonium salts are more efficient catalysts than strong bases.

The invention provides a method for forming aldol condensation products, comprising the step of reacting at least one aldehyde or ketone starting material in the presence of an inorganic ammonium salt, or an aqueous or organic solution prepared from such inorganic salt, at a temperature range of from about 0° C. to about 120° C. and at a pressure range of from about 1 atm to about 1000 atm for a sufficient time to form the aldol condensation products.

In one embodiment, the aqueous inorganic ammonium solution is prepared from an inorganic salt which is chosen from the group comprising NH₄F, (NH₄)₂SO₄, NH₄NO_{3,} NH₄Cl, NH₄Br, NH₄I, NH₄CO_{3,} (NH₄)HSO_{4,} (NH₄)HCO_{3,} and any other inorganic salts containing NH₄⁺cations.

The reactions can take place in solution, or in a heterogenous system with liquid and solid phases. Homogeneous liquid systems can consist of inorganic ammonium solutions of concentrations up to saturation. As examples of suitable concentrations are; NH₄F at 2 M, and (NH₄)₂SO₄ at 3.6 M, both in water. Other examples of conditions are given in Tables 1-3.

Aqueous solutions of the inorganic ammonium salts are preferred, but It is also possible to use the inorganic ammonium salts in organic solutions, such as DMSO, DMF, NMP, THF, Toluene, CH₂Cl₂.CH₃CN.

The inorganic ammonium salts can also be used as catalysts in heterogeneous catalysis, i.e. the catalysis is in a different phase to the reactants. In this case, the inorganic ammonium salt may be in solid phase. For instance, the inorganic ammonium salt can be present beyond its saturation concentration in the solutions described above.

The pH of the solutions does not need to be controlled. Non-buffered aqueous inorganic ammonium salts solutions have a pH between 4 and 7. Example: (NH₄)₂SO₄ 3.6 M, pH=4.9; (NH₄)Cl 4 M, pH=4.6; (NH₄)F 1 M, pH=7.5 (see also Table 3). These mild values of pH constitute both a strong environmental advantage and a strong synthetic advantage of the present invention over currently used catalysts: inorganic ammonium salts catalysts avoid the acid/base wastes produced by current catalysts and can be used with pH-sensitive compounds or in pH-sensitive synthetic methods. The inorganic ammonium salts can also be used under any buffered conditions over the range pH=4-8. Their catalytic efficiency varies over this range, depending on the inorganic ammonium salt and on the starting carbonyl compound, but remains competitive: for instance, with ammonium sulfate, the reaction of acetone is more efficient at lower pH (˜4-5), but the reaction of acetaldehyde is more efficient at higher pH (7-8) (see Table 3).

In a further embodiment, the method is performed at a temperature between about 25° C. and about 70° C.

In a further embodiment, a commercially important application is the self-aldol condensation of acetone. For acetone, the inorganic ammonium salts are more efficient catalysts than the strong base catalysts commonly used (see Table 1). The reaction of acetone produces mesityl oxide (4-methyl-3-penten-2-one), which, upon subsequent hydrogenation, produces Methyl isobutyl Ketone (MIBK, 4-methyl-2-pentanone). This process is carried out in thousands of tons/year and is followed by the treatment of large volumes of strongly basic wastes. Using environmentally-friendly inorganic ammonium salts such as ammonium sulfate, (NH₄)2SO4, would therefore represent a significant cost reduction for this production.

In a further embodiment, the condensation products are purified after completion of the reaction by: (a) separating the condensation products from the catalyst; and

(b) recovering the ionic liquid medium and the catalyst.

Depending on the choice of the inorganic ammonium salt, reactants, substrates and other conditions such as temperature, pressure or pH, a reaction environment can be designed to accommodate the catalysis and the separation of a chemical process in the most efficient way.

Any aldehyde or ketone may be used as starting material in the present case. Alternatively, an Aldol II reaction involves reacting two different aldehyde or ketone starting materials to produce the condensation products. The preferred aldehyde or ketone starting materials utilized in forming the aldol condensation products are one or more aldehydes or ketones having the formula RC(O)R′, wherein R is a hydrogen atom or a straight-chain, branched or cyclic aliphatic group having from 1 to 10 carbon atoms, preferably from 1 to 6 carbon atoms, and most preferably from 1 to 4 carbon atoms.

In a further embodiment, a commercially important application of cross- aldol condensation (Aldol II) is the reaction of formaldehyde with a second aldehyde starting to form a neopolyol. In practice, several intermediate aldol addition steps involving formaldehyde and a final crossed Cannizzaro step take place before producing the neopolyol product. The most preferred second aldehyde starting materials are acetaldehyde, propionaldehyde, n-butyraldehyde and isobutyraldehyde, which, when reacted with formaldehyde, produce the respective neopolyol products pentaerythritol, trimethylol ethane, trimethylol propane and neopentyl glycol.

The ketone and aldehyde starting materials can be used at concentrations up to saturation. An example of a suitable concentration range is between 1 M and 10 M starting material, but even more concentrated solutions can be used, as long as the starting materials are soluble.

The aldehydes and ketones can be saturated or unsaturated aliphatic compounds and can be substituted as long as an a-hydrogen is present in the acceptor compound. Exemplary aliphatic aldehydes include formaldehyde (plus another carbonyl compound since it can act only as a donor), acetaldehyde, propionaldehyde, butyraldehyde, valeraldehyde, dodecanal, octadecanal, 2-ethylhex-2-enal, crotonal, hex-2-enal, 2-ethylbut-2-enal, vinylcrotonal, 2-methylpropanal, and the like. Exemplary cycloaliphatic aldehydes include cyclohex-3-enyl aldehyde.

Representative aliphatic ketones include acetone, methyl ethyl ketone, dibutyl ketones, methyl isobutyl ketone, methyl isoamyl ketone, mesityl oxide, 2-methylnon-5-en-4-one, and the like. Although not necessarily needed, organic solvents may be used in these aldol reactions. For this purpose, hydrocarbons having five to twelve or more atoms such as pentane, decane, hexane, and the like, may be used.

The above examples should only be construed as such and should not be interpreted as limiting the scope of the invention.

The above-referenced reactions for forming aldol condensation products may be generally carried out at a pressure of from about 1 atm (atmospheric pressure) to about 1000 atm (elevated pressure). The reaction can be carried out over a wide range of temperatures and is not particularly limited. Usually the reaction temperature is within the range of from about −20° C. to 120° C., more typically within the range of from 10° C. to 100° C., or 25° C. to 100° C., such as from 25° C. to 70° C.

The duration of the aldol condensation reaction is not critical and should normally be continued until the desired amount of product has been formed. The formation of product can be monitored and quantified by using various analytical methods such as spectrometry by measuring absorbance of light, for example using a UV-vis spectrometer. Another method for measuring the amount of product is by using mass spectrometry, such as High Resolution Mass Spectrometry (HRMS) or various chromatographic methods, such as gas or liquid chromatography. Nuclear magnetic resonance (NMR) may also be used. Other methods form monitoring this are well known to the skilled person.

In the present invention, the catalyst can be recycled by way of methods known in the art and applied as reaction medium to form additional aldol condensation products. The catalyst may also be recycled for use in other reactions.

The following non-limiting examples are provided in order to further demonstrate the various embodiments and advantages of some forms of the present invention

### Examples

General Conditions: 4 ml of aqueous salt solutions (see tables) continuously stirred and protected from light.

Reactant concentration: Acetaldehyde: 0.1-0.5 M; acetone: 0.5-1 M.

Temperature=25 C (room T) or 35 C.

The degree of conversion of the reaction was determined by measuring the consumption of reactant, for acetone, and the formation of the products, for acetaldehyde by UV-visible absorption: acetone monitored at 269 nm, acetaldehyde product (2,4,6-octatrienal) monitored at 320 and 350 nm.

TABLE 1
Comparison between commonly used catalysts and the catalysts
described in this invention for different aldehydes and ketone
(all solutions in water)

| | Acetaldehyde, 25 C. | Acetone, 35 C. |
|---|---|---|
| Catalyst | Conversion (%) at 12 h | Conversion (%) at 3 days |
| H2SO4 12 M | 2% | small (<1%) |
| NaOH 1 M | >80% | 4% |
| Na₂CO₃ 1 M | 82% | 16% |
| (NH₄)₂SO₄ 3.6 M | 10% | 73% |
| (NH4)F 2 M | 23% | 10% |

TABLE 2
Comparison of the catalytic efficiencies of the various inorganic
ammonium salt catalysts described in this invention for the
reaction of acetaldehyde (all solutions in water)

| | Conversion (%) |
|---|---|
| Catalyst/Temperature | at 12 h |
| (NH₄)₂SO₄ 3.6 M/25 C. | 10% |
| (NH₄)F 2 M/25 C. | 23% |
| (NH₄)HSO₄ 3 M/35 C. | 10% |
| NH₄Br 2.5 M/35 C. | 19% |
| NH₄Cl 4 M/35 C. | 30% |
| (NH₄)NO₃ 4 M/35 C. | 35% |

TABLE 3
Effect of pH on the catalytic efficiencies of the various inorganic
ammonium salt catalysts described in this invention for
acetaldehyde and acetone (all solutions in water)

| | | Conversion (%) after 3 days | |
|---|---|---|---|
| | | Acetone | Acetaldehyde |
| Catalyst | pH | (35° C.) | (20° C.) |
| H₂SO₄ 12 M | −3.8 | small | 12.6% |
| NaOH 1 M | 14 | 3% | >97% |
| Na₂CO₃ 1 M | 11.8 | 16% | 95% |
| (NH₄)₂SO₄ 3.5 M, non buffered | 4.9 | 73% | 6% |
| (NH₄)₂SO₄ 3.5 M, buffered | 5.5 | 63% | 12% |
| (NH₄)₂SO₄ 3.5 M, buffered | 6 | 46% | 38% |
| (NH₄)₂SO₄ 3.5 M, buffered | 7 | 43% | 66% |
| (NH₄)₂SO₄ 3.5 M, buffered | 8 | 29% | 72% |

1. A method for forming aldol condensation products, comprising the step of reacting at least one aldehyde or ketone starting material in the presence of an inorganic ammonium salt, or an aqueous or organic solution prepared from such salt, at a temperature range of from about 0° C. to about 120° C. and at a pressure range of from about 1 atm to about 1000 atm for a sufficient time to form aldols, which optionally are dehydrated.

2. The method of claim 1 wherein the aqueous inorganic ammonium solution is prepared from an inorganic salt which is chosen from the group comprising NH₄F, (NH₄)₂SO₄, NH₄NO₃, NH₄Cl, NH₄Br, NH₄I, NH₄CO₃, (NH₄)HSO₄, (NH₄)HCO₃, and any other inorganic salts containing NH₄⁺cations.

3. The method according to claim 1, wherein the temperature is between about 25° C. and about 70° C.

4. The method according to claim 1, wherein the starting material is acetone, in particular, when used for the production of Methyl Isobutyl Ketone (MIBK).

5. The method according to claim 1 further comprising the steps of:
(a) separating the condensation products from the catalyst; and
(b) recovering the ionic liquid medium and the basic catalyst.

6. The method according to claim 2, wherein the temperature is between about 25° C. and about 70° C.
